(19) United States
(12) Patent Application Publication
(10) Pub. No.: US 2014/0114107 A1
(43) Pub. Date: Apr. 24, 2014

(54) USE OF HYDROCARBON DILUENTS TO ENHANCE CONVERSION IN A DEHYDROGENATION PROCESS AT LOW STEAM/OIL RATIOS

(71) Applicant: Lummus Technology Inc., Bloomfield, NJ (US)

(72) Inventor: Ajaykumar Chandravadan Gami, East Brunswick, NJ (US)

(73) Assignee: LUMMUS TECHNOLOGY INC., Bloomfield, NJ (US)

(21) Appl. No.: 14/052,949

(22) Filed: Oct. 14, 2013

Related U.S. Application Data
(60) Provisional application No. 61/717,772, filed on Oct. 24, 2012.

(51) Int. Cl. C07C 5/333

(52) U.S. Cl. CPC C07C 5/333
USPC 585/441; 422/187

(57) ABSTRACT

A process for preparing styrene via the catalytic dehydrogenation of ethylbenzene, comprising recirculation of reaction byproducts to the initial reaction stream as an oil based diluent, providing an effective means for reducing the steam to oil ratio required to operate the catalytic dehydrogenation reactor.

### CROSS-REFERENCE TO RELATED APPLICATIONS

This application, pursuant to 35 U.S.C. §119(e), claims priority to U.S. Provisional Application Ser. No. 61/717,772, filed Oct. 24, 2012, which is herein incorporated by reference in its entirety.

### FIELD OF THE DISCLOSURE

Embodiments disclosed herein relate generally to a process for the production of styrene monomers by the catalytic dehydrogenation of ethylbenzene. More specifically, embodiments disclosed herein relate to catalytic dehydrogenation of ethylbenzene at lower overall weight ratios of water to ethylbenzene (lower overall weight ratios of steam to oil) by recycling a portion of benzene and toluene present in the dehydrogenation effluent to the reactant feed stream as an oil-based diluent.

### BACKGROUND

The production of styrene by the catalytic dehydrogenation of ethylbenzene may be performed as illustrated by the process of FIG. 1. Feed stream 2, which includes ethylbenzene and primary steam, is combined with superheated steam from 6 and fed into a dehydrogenation reactor 12, which contains any appropriate solid-phase dehydrogenation catalyst. The effluent from reactor 12 is then cross-exchanged with superheated steam 8 and introduced to a second dehydrogenation reactor 14. Following the dehydration reaction, the effluent from reactor 14 contains a mixture of styrene monomer, unreacted ethylbenzene, benzene, and toluene. The effluent is then passed through the a series of waste heat exchangers 4, fed by vaporized feed stream 2 and steam 5. Prior to offgas processing, effluent 15 is heat exchanged against cold water at 16 and 17.

The offgas component 36 is processed in offgas processing zone 26. In zone 26, the offgas 36 is compressed and passed through a flux oil scrubber 38 and a flux oil stripper 40 (stripped with steam 28). The non-codensables are recovered as offgas stream 30, hydrocarbons are recycled from stripper 40 via overhead line 31, and hydrocarbon condensates 32 are collected and returned for further processing along with dehydrogenation effluent 18.

The dehydrogenation effluent 18 is collected in a phase separator 19, which isolates the crude styrene-containing product mixture 20 from the aqueous fraction 22. The aqueous fraction 22 is distributed to a skimming tank for recovery of dissolved hydrocarbons and volatile organics, and the crude styrene product 20 is fractionated to obtain a purified styrene product, such as using multiple distillation columns (not shown).

In commercial dehydrogenation processes, such as those described above with respect to FIG. 1, vaporized ethylbenzene is placed into contact with a fixed catalyst bed in the presence of steam, converting a portion of the ethylbenzene to styrene monomer and hydrogen gas. The dehydrogenation of ethylbenzene to form styrene is an endothermic equilibrium reaction, which limits the overall conversion of ethylbenzene because of the reversible nature of the process. As an added concern, the production of styrene monomer occurs simultaneously alongside various side reactions, such as the pyrolytic cracking of ethylbenzene to benzene and toluene, and the oligomerization of styrene monomers to form insoluble residues. While the latter can be suppressed with polymerization inhibitors, cracking must be limited by the reduction of reactor temperatures.

However, due to the endothermic nature of the conversion of ethylbenzene to styrene, the temperature drops rapidly across the catalyst bed of the reactor, reducing or eliminating catalyst activity, leading to decreased production of styrene monomer. Within the field several approaches have been applied to overcome this limitation, including the use of multiple reactor stages with interstage heating. A typical application of interstage heating in multi-reactor setups is the use of indirect heating with steam to restore dehydrogenation effluent to reaction temperature prior to introduction to subsequent reactors. Thus, the inlet temperatures of the reactor are kept at range that is high enough to initiate catalytic conversion, but low enough to avoid excessive loss of ethylbenzene to decomposition reactions.

The use of steam is widely known in the field as a method of introducing at least some of the heat needed to initiate conversion of ethylbenzene to styrene. Steam also acts as a diluent, reducing the partial pressure of the styrene and hydrogen within the reactor and shifting the reaction equilibrium towards the production of styrene. Steam within the reactor also functions as a means of extending the life of the catalyst by removing deposits from reaction surfaces.

The mass steam to oil ratio, i.e., the ratio of steam to ethylbenzene (“oil”) contained in a feed stream on a weight basis (the S/O ratio), is an important factor in the dehydrogenation of ethylbenzene. In commercial applications, reducing the S/O ratio is desirable, because of the costs associated with energy consumption in the vaporization process. Furthermore, excessive use of steam dilutes the reaction mixture, reducing reactor capacity and negatively affecting the overall styrene output of the system. Thus, there have been ongoing efforts in the field to develop dehydrogenation catalysts with enhanced activity under reduced S/O ratios.

Many commercially available catalysts operate at reduced S/O ratios, from about 1.3-1.7, with some catalysts capable of operating at a S/O ratio as low as about 1.0. However, while improvements in the catalysts have reduced the need for lower partial pressures and continuous decoking of the catalysts, because of the multiple roles steam plays in the reaction, further limiting steam feed (i.e., further lowering the S/O ratio) presents other challenges.

### SUMMARY OF INVENTION

In one aspect, embodiments disclosed herein relate to a process for the dehydrogenation of ethylbenzene. The process may include: (a) passing a mixture of ethylbenzene, steam, and an oil-based diluent comprising at least one of benzene and toluene through a catalytic dehydrogenation reactor to produce a dehydrogenation effluent comprising unreacted ethylbenzene, styrene monomer, benzene, toluene and water; (b)separating the dehydrogenation effluent to recover a water fraction and a hydrocarbon fraction comprising ethylbenzene, styrene monomer, benzene, and toluene; (c) fractionating the hydrocarbon fraction to recover a styrene fraction and one or more fractions comprising ethylbenzene, benzene, and toluene; and (d) returning at least a portion of benzene, toluene, or a combination thereof in the one or more fractions recovered in step (c) to step (a) as the oil-based diluent.

In another aspect, embodiments disclosed herein relate to a process for the dehydrogenation of ethylbenzene. The process may include: (a) passing a mixture of ethylbenzene, steam, and an oil-based diluent comprising at least one of toluene and benzene through a catalytic dehydrogenation reactor to produce a dehydrogenation effluent comprising unreacted ethylbenzene, styrene monomer, benzene, toluene and water; (b) separating the dehydrogenation effluent to recover a water fraction and a hydrocarbon fraction comprising ethylbenzene, styrene monomer, benzene, and toluene; (c) separating the hydrocarbon fraction to recover a styrene fraction and a fraction comprising ethylbenzene, benzene, and toluene; and (d) recycling at least a portion of the fraction comprising ethylbenzene, benzene, and toluene to step (a) as an oil-based diluent.

In yet another aspect, embodiments disclosed herein relate to a system for the dehydrogenation of ethylbenzene. The system may include (a) a conduit for feeding a mixture of ethylbenzene, an oil based diluent comprising at least one of benzene and toluene, and steam to a catalytic dehydrogenation reactor to produce a dehydrogenation effluent comprising unreacted ethylbenzene, styrene monomer, benzene, toluene and water; (b) a first separation system for separating the dehydrogenation effluent to recover a water fraction and a hydrocarbon fraction comprising ethylbenzene, styrene monomer, benzene, and toluene; (c) a second separation system for fractionating the hydrocarbon fraction to recover a styrene fraction and one or more fractions comprising ethylbenzene, benzene, and toluene; and (d) a conduit for feeding at least a portion of the one or more fractions comprising ethylbenzene, benzene, toluene, or a combination thereof to the dehydrogenation apparatus of step (a) as the oil-based diluent.

In processes where the total amount of steam is reduced, temperature drops quickly as the reaction proceeds in the reactor, due to the loss of the heat previously provided by the steam fraction. At lower temperatures current catalysts exhibit reduced dehydrogenation efficiency, lowering styrene yields, leading to increased production times in order to generate the same amount of styrene produced at higher S/O ratios, thereby eliminating the cost benefit of reducing steam input. Catalyst performance is also compromised by the absence of diluent, shortening the catalyst lifetime due to poisoning by the buildup of hydrocarbon deposits.

The processes and systems described herein advantageously add oil-based diluents into the reactant feed that provide the additional heat necessary to increase reactor temperatures and styrene yield in reduced steam conditions. By recycling a fraction of the benzene and toluene generated following the removal of styrene as disclosed herein, the dehydrogenation reactor can advantageously be operated at a steam/oil ratio of 1.0 or below.

As a diluent, benzene and toluene are stable at higher reactor temperatures, are compatible with the ethylbenzene reactant, and reduce the further production of benzene and toluene byproducts in accordance with Le Chatelier's Principle. By introducing vaporized diluent into the reactant feed, the temperature remains high enough to preserve catalyst activity, promoting complete conversion to products, and overcoming the primary limitations of the reduction of the S/O ratio. Other aspects and advantages of the invention will be apparent from the following description and the appended claims.

### BRIEF DESCRIPTION OF DRAWINGS

FIG. 1 is a simplified process flow diagram of a prior art process for the dehydrogenation of ethylbenzene to form styrene.

FIG. 2A and 2B are simplified process flow diagrams of a process for the production of styrene monomer (SM) according to embodiments disclosed herein.

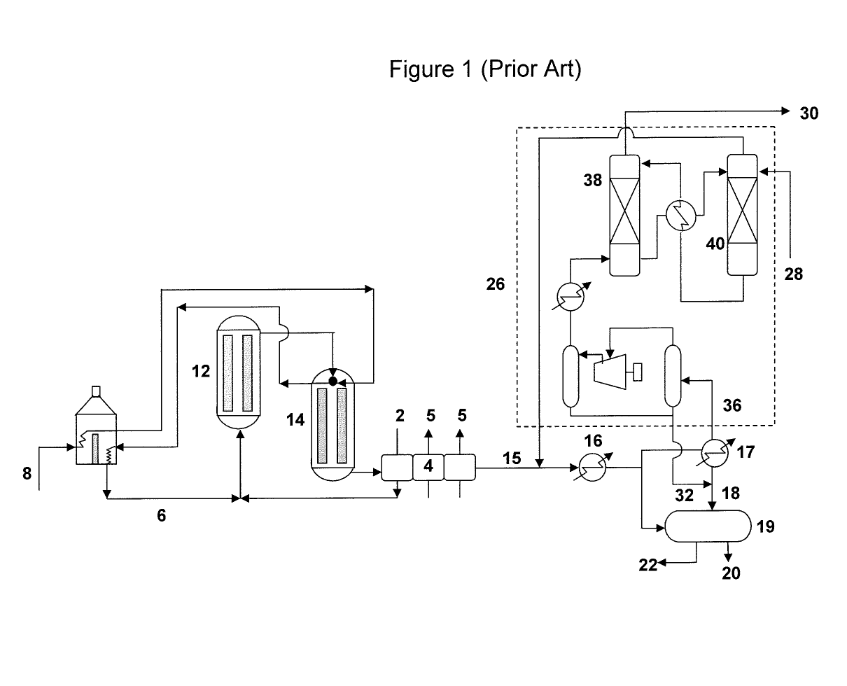

### DETAILED DESCRIPTION

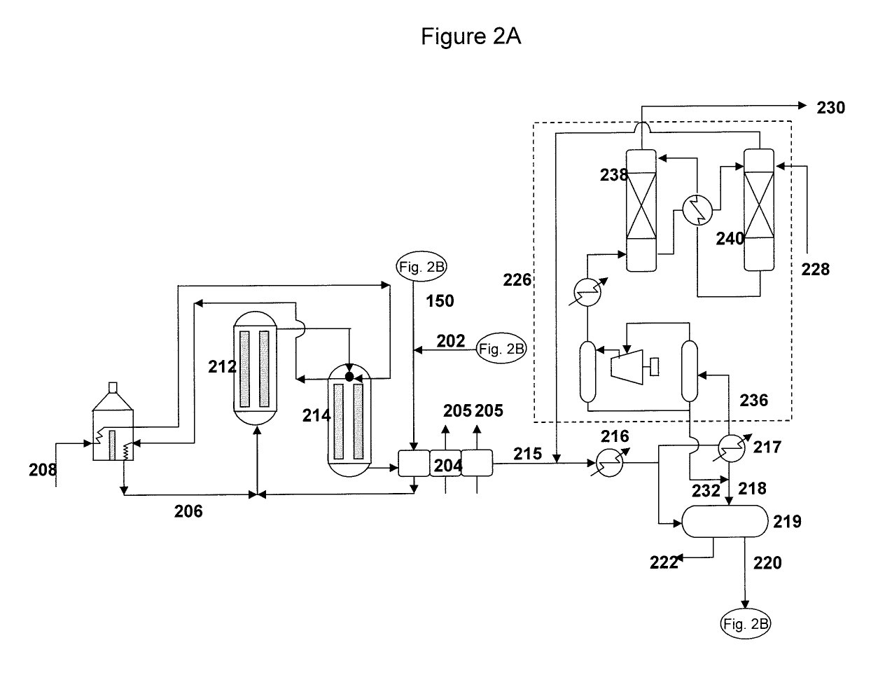

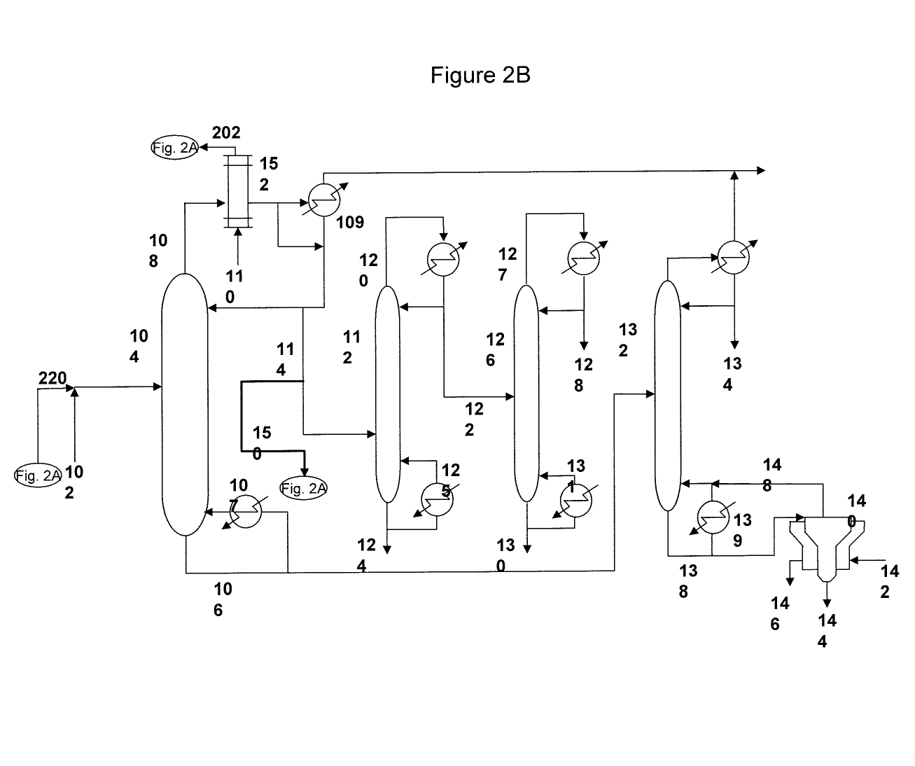

Referring to FIG. 2A, a method for the production of styrene from the catalytic dehydrogenation of ethylbenzene is illustrated. Feed stream 202, which includes ethylbenzene and primary steam, is combined with oil based diluent 150 and superheated steam 206 and fed into a dehydrogenation reactor 212, which contains any appropriate solid-phase dehydrogenation catalyst.

Reactor setup can vary from multiple beds contained in a single reactor, or single beds in multiple reactors, or a mixture of these arrangements. As illustrated, two reactors 212, 214 in series are used for the desired conversion. The effluent from reactor 212 is cross-exchanged with superheated steam 208 and introduced to a second dehydrogenation reactor 214 for continued reaction. Following the dehydration reaction, the effluent from reactor 214 contains a mixture of styrene monomer, unreacted ethylbenzene, benzene, and toluene. The effluent is then passed through a series of waste heat exchangers 204, fed by vaporized feed stream 202 and steam 205. Prior to offgas processing in zone 226, cooled effluent 215 is heat exchanged against cold water in exchangers 216, 217 to further reduce the temperature of the effluent and condense the hydrocarbons.

In offgas processing zone 226, the offgas fraction 236 (uncondensed reactor effluent) is compressed and processed through a flux oil scrubber 238 and a flux oil stripper 240. The non-condensable lights are evacuated via stream 230, recycle hydrocarbons (C6's-C8's or higher, for example) are recovered in the stripper overheads and combined with steam 228 for combination with effluent 215 and further processing. The recirculating flux oil may be recovered as a bottoms from stripper 240 and recycled as the scrubbing fluid in flux oil scrubber 238 (accumulated heavies may be purged, although not shown). Condensates 232 are also collected and returned for further processing along with the dehydrogenation effluent 218.

The dehydrogenation effluent 218 is collected in a phase separator 219, which isolates the crude styrene-containing product mixture 220 from the aqueous fraction 222. Aqueous fraction 222 is distributed to a skimming tank for the recovery of dissolved hydrocarbons and volatile organics, and the crude styrene hydrocarbon mixture 220 is processed to obtain a purified styrene as illustrated in FIG. 2B.

Referring now to FIG. 2B, following removal of offgas and aqueous condensates, the crude styrene-containing product 220 is mixed with a polymerization inhibitor 102 and fed into a first distillation column 104 for separation of the dehydration effluent into a styrene-rich bottoms 106 and an overheads fraction 108, including the unreacted ethylbenzene, benzene and toluene. In some embodiments, overheads 108 may be used to partially vaporize reactant feed stream 110 via cross exchange with overheads fraction 108 in exchanger 152.

Overheads fraction 108 is then condensed (or further condensed) via heat exchanger 109, a portion of which is returned to column 104 as reflux. The remaining overhead condensate is recovered via flow line 114.

A portion of the ethylbenzene, benzene, and toluene in stream 114 is used as oil based diluent 150 and combined with the ethylbenzene/water reactant feed stream 110/202 (as shown in FIG. 2A). Ethylbenzene in stream 150 is a recycled reactant, while the toluene and benzene are an oil-based diluent. Oil-based diluent 150 may be combined with the ethylbenzene reactant stream prior to vaporization, and/or may be vaporized separately prior to admixture with reactant stream 202.

The remainder of stream 114 is then fed to distillation column 112 for further separation. In column 112, ethylbenzene is recovered as a bottoms fraction 124 and the lower boiling benzene and toluene reaction by-products are recovered as an overheads fraction 120. The ethylbenzene-containing bottoms fraction 124 may be recycled as additional reactant for the dehydrogenation reaction.

Overheads fraction 120 is then condensed, a portion of which is fed via stream 122 to distillation column 126. In column 126, the benzene and toluene are separated, the benzene being recovered as overheads fraction 127 and the toluene product being recovered as bottoms fraction 130. Overheads fraction 127 may then be condensed, a portion being returned to column 126 as reflux, and the remaining being recovered as benzene product stream 128.

Styrene-rich fraction 106 is passed from column 104 to a styrene purification column 132 for separation of the styrene product from oligomers and other heavies. The substantially pure styrene monomer product is recovered as an overheads, condensed and returned as reflux to the column or isolated as styrene product stream 134. The bottoms fraction 138, including styrene, oligomerized styrene, and tars, are vaporized and returned to the column as reboil, or transferred to thin film evaporator 140. Within thin film evaporator 141, steam 142 vaporizes the low boiling volatiles, such as styrene monomer, which are recovered and returned to column 132, while oligomers and tar exit as heavies stream 144.

As described above, oil-based diluent may be provided to the dehydrogenation reactors via flow stream 150. Oil-based diluent may alternatively or additionally be obtained from other streams along the distillation train. For example, it may be advantageous to recycle a benzene and/or toluene-containing vapor draw from one or more of the distillation columns in order to obviate the need for vaporization prior to admixture with the ethylbenzene feed stream. For example, in FIG. 2B, a vapor draw may be taken from streams 108, 120, 127, or a combination thereof and recycled to reactant stream 202 as an oil-based diluent.

In yet other embodiments, benzene and/or toluene-containing fractions may be diverted from a liquid stream and vaporized prior to use as a return to the ethylbenzene feed stream. For Example, in FIG. 2B, a fraction of the liquid streams 114, 124, 122, 130, 128, 134, or a combination thereof may be recycled as an oil-based diluent.

When a portion of a liquid draw is returned as the oil-based diluent, such benzene and/or toluene-containing fractions may be vaporized or partially vaporized by recovering heat from a process stream associated with the distillation column(s) prior to admixture with the ethylbenzene feed stream as an oil-based diluent. Suitable heat sources would include excess steam from heat exchangers 107, 125, 131, 139, steam condensate stream 146, cross exchange with product streams 106, 124, 130, 144, or heat exchange with appropriate overheads streams.

In some embodiments, the amount of oil-based diluent in the feed may be in the range from about 5 wt. % to about 50 wt. %, based on the total feed to the reactors (including steam, ethylbenzene, and the oil-based diluent, which may include toluene, benzene, or a combination thereof). In other embodiments, the amount of oil-based diluent in the feed may be in the range from about 10 wt. % to about 40 wt. %, based on the total feed to the reactors; from about 15 wt. % to about 35 wt. % in other embodiments; and from about 20 wt. % to about 30 wt. % in yet other embodiments.

Using oil-based diluents according to embodiments disclosed herein, the catalytic dehydrogenation reactors may be operated at a steam to oil (EB) ratio (S/O ratio) of less than 1.0; in other embodiments, the S/O ratio may be in the range from about 0.30 to about 0.75; in other embodiments, the S/O ratio may be in the range from about 0.45 to about 0.55; and in yet other embodiments the S/O ratio may be in the range from about 0.48 or 0.49 to about 0.51 or about 0.52, such as about 0.5.

The operating temperature of the dehydrogenation reactor should be in a range from about 500° C. to about 1000° C., preferably in a range from about 550° C. to about 750° C., and more preferably in a range from about 600° C. to about 650° C. In some embodiments, the dehydrogenation reactor pressure may vary from about 40 to about 80 kPa. It is important that sufficient pressure be maintained at the reactor inlet to overcome the pressure drop through the catalyst bed(s) contained in the reactor vessel or in separate vessels if each such bed is contained in a separate reactor. Suitable catalysts include palladium oxide, platinum metal , supported palladium, molybdenum-bismuth oxide, ferrous oxide-potassium oxide, other metal oxides and/or sulfides, including those of calcium, lithium, strontium, magnesium, beryllium, zirconium, tungsten, molybdenum, titanium, hafnium, vanadium, aluminum, chromium, copper, and mixtures of two or more including chromia-alumina, alumina-titania, alumina-vanadia, etc. Dehydrogenation may be conducted at atmospheric pressure, although in some cases, subatmospheric or superatmospheric pressure may be desirable.

As a diluent, benzene and toluene are stable at higher reactor temperatures, and are compatible with the ethylbenzene reactant. By introducing vaporized diluent into the reactant feed, the temperature remains high enough to preserve catalyst activity, promoting complete conversion to products and overcoming the primary limitations of the reduction of the S/O ratio, the oil-based diluent acting as an additional heat source.

Recycled benzene and toluene in the diluent stream may also increase the efficiency of the catalytic conversion. In accordance with Le Chatelier's principle the increased concentration of benzene and toluene may shift the reaction equilibrium to favor the production of styrene monomer, resulting in an overall decrease in the production of byproducts.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A process of producing styrene from the catalytic dehydrogenation of ethylbenzene, comprising the steps of:
(a) passing a mixture of ethylbenzene, steam, and an oil-based diluent comprising at least one of benzene and toluene through a catalytic dehydrogenation reactor to produce a dehydrogenation effluent comprising unreacted ethylbenzene, styrene monomer, benzene, toluene and water;
(b) separating the dehydrogenation effluent to recover a water fraction and a hydrocarbon fraction comprising ethylbenzene, styrene monomer, benzene, and toluene;
(c) fractionating the hydrocarbon fraction to recover a styrene fraction and one or more fractions comprising ethylbenzene, benzene, and toluene;
(d) returning at least a portion of benzene, toluene, or a combination thereof in the one or more fractions recovered in step (c) to step (a) as the oil-based diluent.

2. The process of claim 1, comprising operating the catalytic dehydrogenation reactor of step (a) at a steam to oil ratio of less than 1.

3. The process of claim 1, comprising operating the catalytic dehydrogenation reactor of step (a) at a steam to oil ratio in the range from about 0.35 to about 0.65.

4. The process of claim 1, further comprising vaporizing ethylbenzene prior to the catalytic dehydrogenation of step (a).

5. The process of claim 1, wherein the one or more fractions comprising ethylbenzene, benzene and toluene include at least one of: an ethylbenzene-benzene-toluene fraction; a benzene-toluene fraction; an ethylbenzene fraction; a toluene fraction; and a benzene fraction.

6. The process of claim 5, wherein a portion of at least one of the ethylbenzene-benzene-toluene fraction, the benzene-toluene fraction, the benzene fraction, and the toluene fraction is used as the portion returned in step (d).

7. The process of claim 6, wherein the portion returned comprises a least one of a vapor draw, a liquid draw, an overhead product, and a bottoms product from a distillation column used in fractionating step (c).

8. The process of claim 7, wherein the portion returned comprises a liquid, the process further comprising vaporizing the portion returned.

9. The process of claim 8, wherein the vaporizing comprises heat exchange with a process stream associated with the distillation column(s) used in fractionating step (c).

10. A process of producing styrene from the catalytic dehydrogenation of ethylbenzene, comprising the steps of:
(a) passing a mixture of ethylbenzene, steam, and an oil-based diluent comprising at least one of toluene and benzene through a catalytic dehydrogenation reactor to produce a dehydrogenation effluent comprising unreacted ethylbenzene, styrene monomer, benzene, toluene and water;
(b) separating the dehydrogenation effluent to recover a water fraction and a hydrocarbon fraction comprising ethylbenzene, styrene monomer, benzene, and toluene;
(c) separating the hydrocarbon fraction to recover a styrene fraction and a fraction comprising ethylbenzene, benzene, and toluene;
(d) recycling at least a portion of the fraction comprising ethylbenzene, benzene, and toluene to step (a) as an oil-based diluent.

11. The process of claim 10, further comprising:
separating the fraction containing ethylbenzene, benzene, and toluene to recover an ethylbenzene fraction and a fraction comprising benzene and toluene;
separating the fraction containing benzene and toluene to recover a benzene fraction and a toluene fraction;

12. The process of claim 11, wherein the recycled at least a portion comprises at least one of a portion of the fraction comprising benzene and toluene, a portion of the benzene fraction, and a portion of the toluene fraction.

13. The process of claim 10, comprising operating the catalytic dehydrogenation reactor of step (a) at a steam to oil ratio of less than 1.

14. The process of claim 10, comprising operating the catalytic dehydrogenation reactor of step (a) at a steam to oil ratio in the range from about 0.35 to about 0.65.

15. The process of claim 10, wherein the returned portion in step (d) comprises at least one of a vapor draw, a liquid draw, an overhead product, and a bottoms product from a distillation column used in separating step (c).

16. The process of claim 15, wherein the returned portion comprises a liquid, the process further comprising vaporizing the returned portion.

17. The process of claim 16, wherein vaporizing comprises heat exchange with a process stream associated with the distillation column(s) used in fractionating step (c).

18. An apparatus for producing styrene from the catalytic dehydrogenation of ethylbenzene, comprising:
(a) a conduit for feeding a mixture of ethylbenzene, an oil based diluent comprising at least one of benzene and toluene, and steam to a catalytic dehydrogenation reactor to produce a dehydrogenation effluent comprising unreacted ethylbenzene, styrene monomer, benzene, toluene and water;
(b) a first separation system for separating the dehydrogenation effluent to recover a water fraction and a hydrocarbon fraction comprising ethylbenzene, styrene monomer, benzene, and toluene;
(c) a second separation system for fractionating the hydrocarbon fraction to recover a styrene fraction and one or more fractions comprising ethylbenzene, benzene, and toluene;
(d) a conduit for feeding at least a portion of the one or more fractions comprising ethylbenzene, benzene, toluene, or a combination thereof to the dehydrogenation apparatus of step (a) as the oil-based diluent.

19. The apparatus claim 18, wherein the conduit of (d) for feeding the oil-based diluent includes one or more of:
a flow conduit for feeding at least a portion of an ethylbenzene-benzene-toluene containing fraction;
a flow conduit for feeding at least a portion of a benzene-toluene containing fraction;
a flow conduit for feeding at least a portion of an ethylbenzene fraction;
a flow conduit for feeding at least a portion of a toluene fraction; and
a flow conduit for feeding at least a portion of a benzene fraction.

20. The apparatus of claim 19, further comprising a heat exchanger for vaporizing the oil-based diluent.
